(19) United States
(12) Patent Application Publication
(10) Pub. No.: US 2023/0086618 A1
(43) Pub. Date: Mar. 23, 2023

(54) Improved Systems, And Methods of Solventless Extraction of Cannabinoid Compounds

(71) Applicant: Trichome Alchemy LLC, Arvada, CO (US)

(72) Inventors: Jacob William Myers, Arvada, CO (US); Kerry Kate Conley, Arvada, CO (US)

(21) Appl. No.: 17/799,809

(22) PCT Filed: Mar. 10, 2021
(86) PCT No.: PCT/US2021/021772

§ 371 Date: Aug. 15, 2022

Related U.S. Application Data
(60) Provisional application No. 62/987,719, filed on Mar. 10, 2020.

(51) Int. Cl. A61K 36/185

(52) U.S. Cl. CPC A61K 36/185; A61K 2236/00

(57) ABSTRACT

The inventive technology includes novel systems, methods, and apparatus for the solventless separation and extraction of trichome structures containing short-chain fatty acid phenolic compounds using a novel multi-staged trichome collection array that may be configured to separate and extract trichome structures in cannabinoid-producing plants such as Cannabis.

This International PCT application claims the benefit of and priority to U.S. Provisional Application No. 62/987,719, filed Mar. 10, 2020, the entirety of which is incorporated herein by reference in its entirety.

### TECHNICAL FIELD

The inventive technology is generally related to the field of phytochemical separation and extraction. In particular, the inventive technology includes improved systems, methods, and apparatus for the solventless separation and extraction of trichome structures containing short-chain fatty acid phenolic compounds, such as cannabinoids and terpenes from plant material, including those of the plant family Cannabaceae.

### BACKGROUND

Cannabinoids are a class of specialized compounds synthesized by Cannabis plants, among others. They are formed by condensation of terpene and phenol precursors. The most abundant cannabinoids include: Δ⁹-tetrahydrocannabinol (THC), cannabidiol (CBD), cannabichromene (CBC), and cannabigerol (CBG). Another cannabinoid, cannabinol (CBN), is formed from THC as a degradation product and can be detected in some plant strains. Typically, THC, CBD, CBC, and CBG occur together in different ratios in the various plant strains. These cannabinoids are generally lipophilic, nitrogen-free, mostly phenolic compounds and are derived biogenetically from a monoterpene and phenol, the acid cannabinoids from a monoterpene and phenol carboxylic acid and have a C21 base. Cannabinoids also find their corresponding carboxylic acids in plant products. In general, the carboxylic acids have the function of a biosynthetic precursor. For example, these compounds arise in vivo from the THC carboxylic acids by decarboxylation of the tetrahydrocannabinols Δ⁹- and Δ⁸-THC and CBD from the associated cannabidiol. Cannabinoids are generally classified into two types, neutral cannabinoids and cannabinoid acids, based on whether they contain a carboxyl group or not. It is known that, in fresh plants, the concentrations of neutral cannabinoids are much lower than those of cannabinoid acids. As a result, THC and CBD may be derived artificially from their acidic precursor compounds tetrahydrocannabinolic acid (THCA) and cannabidiolic acid (CBDA) by non-enzymatic decarboxylation.

Notably, cannabinoids are toxic compounds and generally harmful to plant cells. Moreover, cannabinoid synthesis produces toxic by-products. Notably, both CBDA and THCA synthases require molecular oxygen, in conjunction with a molecule of FAD, to oxidize cannabigerolic acid (CBGA). Specifically, two electrons from the substrate are accepted by an enzyme-bound FAD, and then transferred to molecular oxygen to re-oxidize FAD. CBDA and THCA are synthesized from the ionic intermediates via stereoselective cyclization by the enzymes. The hydride ion is transferred from the reduced flavin to molecular oxygen, resulting in the formation of hydrogen peroxide (H₂O₂) and re-activation of the flavin for the next cycle. As a result, in addition to producing CBDA and THCA respectively, this reaction produces hydrogen peroxide which is naturally toxic to the host cell.

Cannabis plants deal with these cellular cytotoxic effects through a process of directing cannabinoid production to extracellular structures. Specifically, cannabinoid biosynthesis is localized in the secretory cavity of the glandular trichomes which are abundant on the surface of the female inflorescence in Cannabis sativa. Trichomes can be visualized as small hairs or other outgrowths from the epidermis of a Cannabis plant. For example, THCA synthase is a water-soluble enzyme that is responsible for the production of THC. For example, THC biosynthesis occurs in glandular trichomes and begins with condensation of geranyl pyrophosphate with olivetolic acid to produce cannabigerolic acid (CBGA); the reaction is catalyzed by an enzyme called geranylpyrophosphate:olivatolate geranyltransferase. CBGA then undergoes oxidative cyclization to generate tetrahydrocannabinolic acid (THCA) in the presence of THCA synthase. THCA is then transformed into THC by non-enzymatic decarboxylation. Prior sub-cellular localization studies using RT-PCR and enzymatic activity analyses demonstrate that THCA synthase is expressed in the secretory cells of glandular trichomes, and then is translocated into the secretory cavity where the end product THCA accumulates. THCA synthase present in the secretory cavity is functional, indicating that the storage cavity is the site for THCA biosynthesis and storage. In this way, the Cannabis plant is able to produce cannabinoids extracellularly and thereby avoid the cytotoxic effects of these compounds. In addition to cannabinoids, trichomes in Cannabis are also the sites of production of other secondary compounds like terpenes, which are responsible for the distinctive aroma of Cannabis.

A wide range of processes to extract phytochemical from plants, such as cannabinoids, are known and taught in the prior art. Typically, non-aqueous solvents-based methods are employed to extract cannabinoids and other phytochemicals from Cannabis plant material. For example, in U.S. Pat. No. 6,403,126 (Webster et al.), cannabinoids, and other related compounds are isolated from raw harvested Cannabis and treated with an organic solvent, typically a petroleum derived hydrocarbon, or a low molecular-weight alcohol to solubilize the cannabinoids for later isolation. This traditional method is limited in that it relies on naturally grown plant matter that may have been exposed to various toxic pesticides, herbicides and the like. In addition, such traditional extraction methods are imprecise resulting in unreliable and varied concentrations of extracted THC. In addition, many Cannabis strains are grown in hydroponic environments which are also not regulated and can result in the widespread contamination of such strains with chemical and other undesired compounds.

In another example, U.S. Pat. App. No. 20160326130 (Lekhram et al.), cannabinoids, and other related compounds are isolated from raw harvested Cannabis using, again, a series of organic solvents to convert the cannabinoids into a salt, and then back to its original carboxylic acid form. Similar to Webster, this traditional method is limited in that it relies on naturally grown plant matter that may have been exposed to various toxic pesticides, herbicides and the like. In addition, the multiple organic solvents used in this traditional process must be recovered and either recycled and/or properly disposed of.

Another traditional method of cannabinoid extraction involves the generation of hash oils utilizing supercritical carbon-dioxide (sCO₂). Under this traditional method, again the dried plant matter is ground and subjected to a sCO₂ extraction environment. The primary extract is initially obtained and further separated. For example, as generally described by CA2424356 (Muller et al.), cannabinoids are extracted with the aid of sCO₂ under supercritical pressure and temperature conditions and by the addition of accessory solvents (modifiers) such as alcohols. Under this process, this supercritical CO2 evaporates and dissolves into the cannabinoids. However, this traditional process also has certain limiting disadvantages. For example, due to the low solubility in supercritical sCO₂, recovery of the cannabinoids of interest is inconsistent. Additionally, any solvents used must be recycled and pumped back to the extractor, in order to minimize operating costs.

Another method utilizes butane to extract cannabinoids, in particular high concentrations of THC, from raw harvested Cannabis. Because butane is non-polar, this process does not extract water soluble by-products such as chlorophyll and plant alkaloids. That said, this process may take up to 48 hours, and as such, is limited in its ability to scale-up for maximum commercial viability. The other major drawback of traditional butane-based extraction processes is the potential dangers of using flammable solvents, as well as the need to ensure all of the butane is fully removed from the extracted cannabinoids.

In an attempt to circumvent the problems associated with solvent-based extraction systems, solventless phytochemical extraction systems have been developed. However, as discussed below, they too suffer from significant technical and cost disadvantages. For example, as outlined in FIG. 17, a traditional method of solventless extraction has been developed that involves manually separating the individual trichome structures from the Cannabis plant material in a cold environment, such as ice water. The separated trichome structures are then manually passed through a series of buckets each containing a lining having a filter in the bottom where the trichome structures may be captured. Typical filters used in this process may generally be referred to as Bubble Bags™. Bubble Bags™ may be formed from a nylon material and placed around a standard sized food grade bucket with the bottom portion being a filter for capturing trichome structures. During this process, pressurized water must be continually passed through the Bubble Bag™ to wash trichome particles, which may generally be referred to as hash particles or hash resin, from the sides of the filter through the mesh screen positioned at the bottom of the bag.

This process must be repeated multiple times as the water containing the separated trichome structures passes through Bubble Bags™ having progressively smaller and smaller filters. The captured trichomes may be removed and further processed to form hash resin for commercial or therapeutic uses. It should be noted that this traditional process is extremely labor intensive and time consuming. Hash resin yield can also be affected by temperature changes during the manual transfer between Bubble Bags™, further limiting the overall effectiveness of this process. In addition, the size of the filters, such as the standard Bubble Bags™, limits their ability to effectively scale production, or form a continuous or semi-continuous closed-loop production system that can be efficiently scaled for commercial purposes. Finally, the inefficient nature of such open-loop small-batch ice-water extraction methods can erode margins making any the products more susceptible to volatility in the Cannabis market.

As demonstrated above, there exists a long-felt need for a cost-effective and efficient technical solution to the problems associated with both solvent, and solventless extraction systems. As will be discussed in more detail below, the current inventive technology overcomes the limitations of these traditional methods while meeting the objectives of a truly cost-effective and effective cannabinoid/hash resin extraction system.

### SUMMARY OF THE INVENTION

On aspect of the inventive technology includes a novel closed-loop trichome separation and extraction system that may be implemented to produce hash resin for commercial and therapeutic uses.

In one preferred aspect, the inventive technology includes a novel multi-staged trichome collection array that may be configured to separate and extract trichome structures containing short-chain fatty acid phenolic compounds, such as cannabinoids, terpenes and other volatile compounds found in cannabinoid-producing plants such as Cannabis.

In another aspect, the inventive technology includes a novel multi-staged trichome collection array that may be configured to include one or more modular separation columns configured to hold one, or a plurality of mesh inserts that are configured to capture trichome structures separated from plant material. In this preferred aspect, each of the mesh inserts may have a discrete mesh, or pore size, allowing the system to capture differentially sized trichome structures that may have unique phytochemical properties. In one preferred embodiment, the mesh inserts may be formed of metal, and in particular food/pharmaceutical grade steel, or other metal that may be approved as part of GMP practices for the extraction and commercial or therapeutic use of trichome structures and Cannabinoids.

In another aspect, the inventive technology includes a novel multi-staged trichome collection array that may be configured to include one or more modular separation columns, a modular separation column configured to secure a plurality of sequentially positioned mesh inserts in series along the length of the column and wherein each mesh insert has a smaller pore size than the prior mesh insert. In one preferred aspect, each sequentially positioned mesh insert may be positioned within a support mesh insert, which may preferably include a metal mesh insert configured to support the mesh insert, while allowing the unrestricted flow of carrier liquid through the column. In one preferred aspect, the support mesh insert may be configured to have a standard mesh, or pore size, which may be larger than the mesh, or pore size of the mesh insert to allow unrestricted flow of carrier liquid through the column.

In one preferred aspect, the inventive technology includes a novel closed-loop multi-staged trichome collection array that may be configured for a vacuum directed flow of biomass, and in particular Cannabis biomass, and a carrier liquid that can separate and extract trichome structures containing short-chain fatty acid phenolic compounds, such as cannabinoids, terpenes and other volatile compounds found in cannabinoid-producing plants such as Cannabis.

In one preferred aspect, the inventive technology includes methods, and systems for a close-loop system for the solventless separation and extraction of trichome structures, and in particular trichome structures from Cannabis. In this preferred aspect, the invention may include a novel system and apparatus for the controlled agitation of Cannabis biomass to remove trichome structures prior to extraction and isolation in the modular separation column. In this aspect, the invention may include a novel multi-directional agitation nozzle configured to generate a controlled rate of agitation and turbulent water-flow within an agitation tank, for example. The controlled agitation allows the trichome structures to be separated from the biomass, while not destroying the plant material.

In one preferred aspect, the inventive technology includes methods, and systems for a close-loop system for the solventless separation and extraction of trichome structures, and in particular trichome structures from Cannabis that is further configured to be recirculated back through the system for further trichome extraction.

Additional aspects of the inventive technology will become apparent from the specification, figures and claims below.

### BRIEF DESCRIPTION OF THE FIGURES

Aspects, features, and advantages of the present disclosure will be better understood from the following detailed descriptions taken in conjunction with the accompanying figures, all of which are given by way of illustration only, and are not limiting the presently disclosed embodiments, in which:

FIG. 1 shows an assembled modular separation column of the invention in one embodiment thereof;

FIG. 2 shows an assembled modular separation column coupled to an exemplary support frame of the invention in one embodiment thereof;

FIG. 3 shows an assembled modular separation column coupled to an exemplary support frame through a plurality of mounting couplers coupled with support frame brackets in one embodiment thereof;

FIG. 4 (A) shows an isolated column coupler in one embodiment thereof; 4 (B) shows an assembled modular separation column of the invention formed by four modular casings coupled together by a series of column couplers as well as an end-cap positioned at the proximal and distal ends of the column and secured by a column coupler in one embodiment thereof; 4 (C) shows an enhanced view of a modular casing coupled with an end-cap by a column coupler as well as a mounting coupler having an insulated covering secured to a terminal modular casing in one embodiment thereof;

FIG. 5 (A) shows a front perspective view of an isolated modular casing in one embodiment thereof; 5 (B) shows a side perspective view of an isolated modular casing in one embodiment thereof; 5 (C) shows a top view of an isolated modular casing in one embodiment thereof;

FIG. 6 (A) shows an isolated mounting coupler having an insulated covering further coupled with a support frame bracket in one embodiment thereof; 6 (B) shows a mounting coupler having an insulated covering secured to a centrally positioned modular casing in one embodiment thereof;

FIG. 7 shows a mixing tank, an optional settling reservoir and feed conduit in one embodiment thereof;

FIG. 8 shows a cross-section of a top portion of a modular separation column having a first mesh insert secured between a proximal end cap and first modular casing in one embodiment thereof;

FIG. 9 shows a cross-section of modular separation column having a plurality of descending mesh insert internally secured within the column in one embodiment in one embodiment thereof;

FIG. 10 shows a mixing tank and pump for recirculation of wastewater from the modular separation column in one embodiment thereof;

FIG. 11 shows a side and top view of a mesh insert in one embodiment thereof;

FIG. 12 shows a 12 (A) perspective, 12 (B) top and 12 (C) side view of a mesh insert having a base and radial extension configured to be secured within modular separation column in one embodiment thereof;

FIG. 13 shows a bottom view of a multi-directional agitation nozzle in one embodiment thereof;

FIG. 14 shows a top view of a multi-directional agitation nozzle in one embodiment thereof;

FIG. 15 shows a top view of a multi-directional agitation nozzle in one embodiment thereof;

FIG. 16 shows a stepwise flow-chart of the improved method solventless extraction of cannabinoids from Cannabis plant material in one embodiment thereof;

FIG. 17 shows a schematic diagram of the improved method solventless extraction of cannabinoids from Cannabis plant material in one embodiment thereof; and

FIG. 18 shows a stepwise flow-chart of the prior art process of solventless extraction of cannabinoids from Cannabis plant material using traditional bag-screening methods.

### DETAILED DESCRIPTION OF THE INVENTION

The inventive technology includes a novel closed-loop multi-staged trichome collection array (1) that may be configured to separate and extract trichome structures containing short-chain fatty acid phenolic compounds, such as cannabinoids, terpenes and other volatile compounds found in cannabinoid-producing plants such as Cannabis.

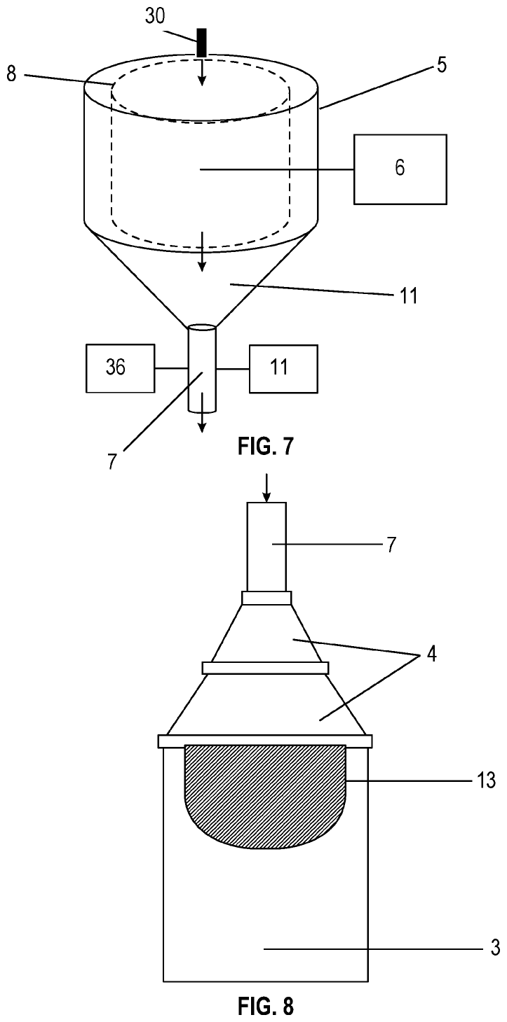

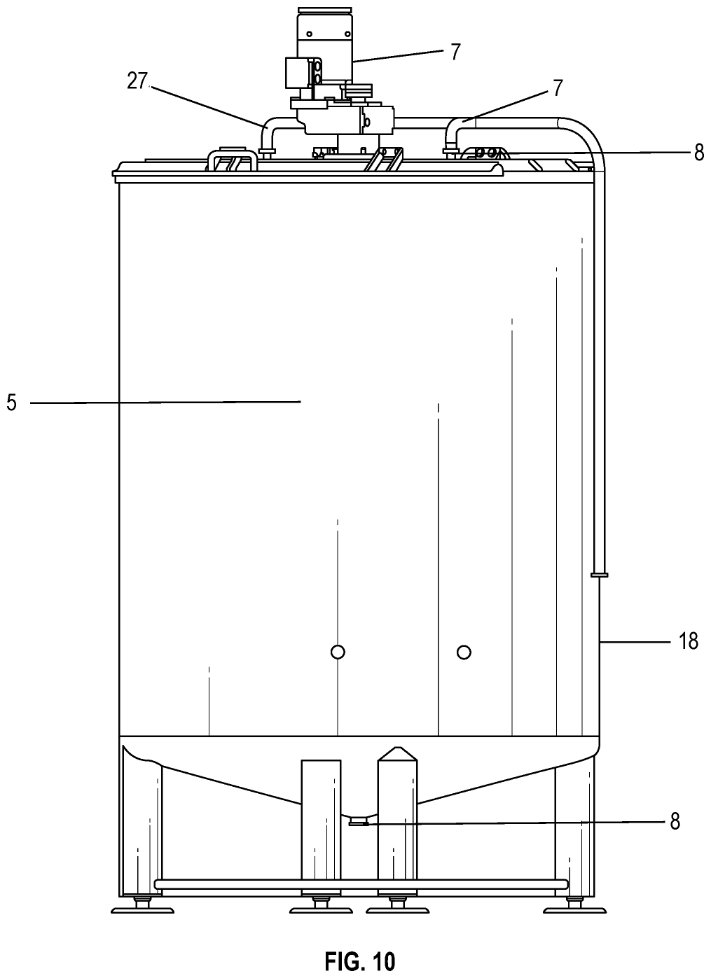

In one embodiment, a multi-staged trichome collection array (1) may include one or more mixing tanks (5). Generally referring to FIGS. 7 and 10, in this preferred embodiment a mixing tank (5) may include a suitable vessel to hold a quantity of plant material to be processed and may further be configured to generate a temperature controlled environment. For example, plant material, and preferably frozen Cannabis plant material, may be positioned in a mixing tank (5) along with a quantity of water and ice to reduce the temperature such that the non-living trichome structures may be more easily separated from the plant material. Notably, the water and/or ice that is used to process the plant material may first undergo one or more purification steps to remove any impurities, additives, trace minerals or other undesired compositions. For example, the water and/or ice that is used to process the plant material may first undergo a process of reverse-osmosis (RO) whereby water molecules are caused to pass through a membrane in response to a natural or artificial gradient and thereby may be purified of the aforementioned impurities.

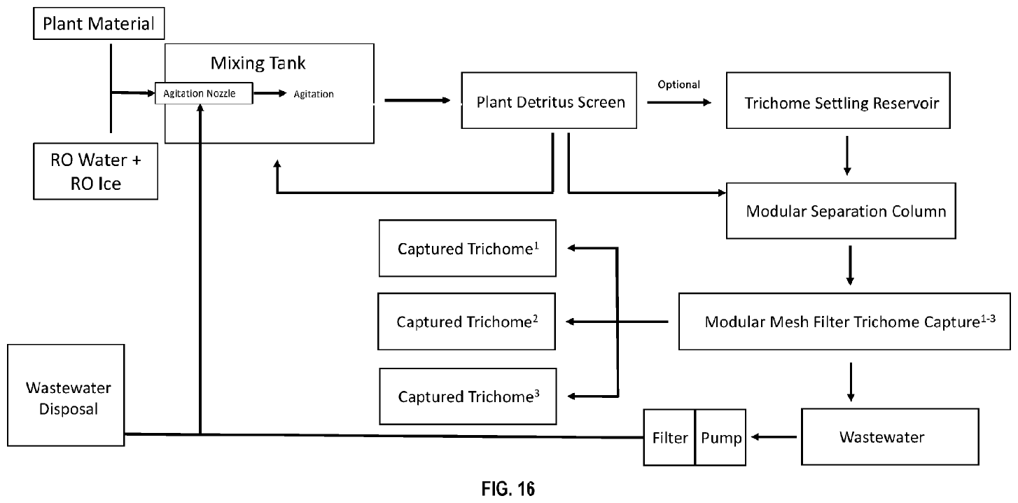

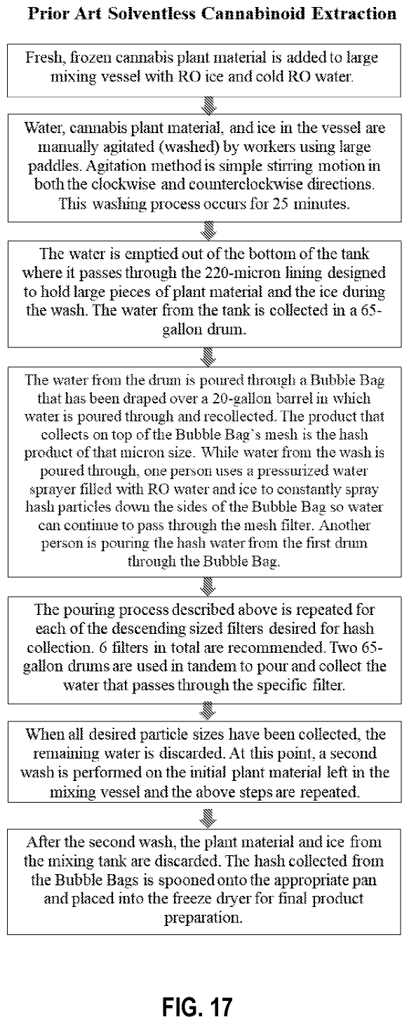

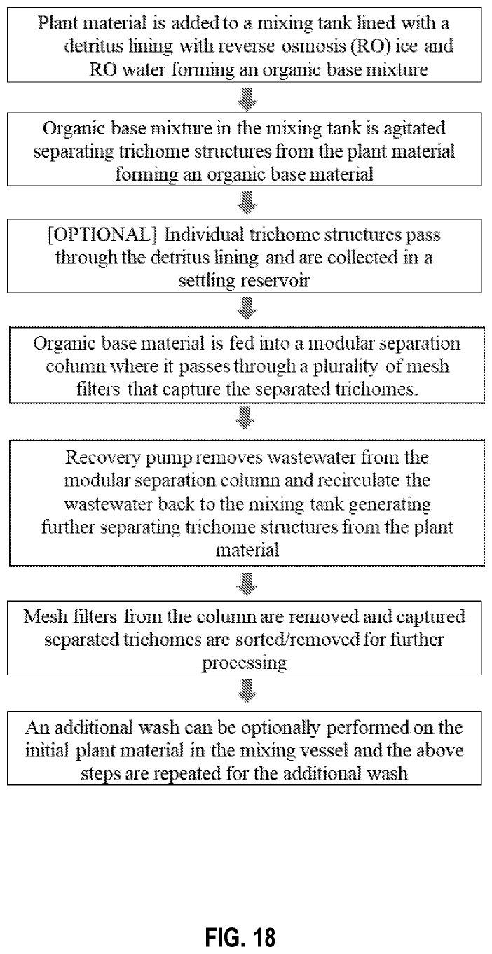

As outlined in FIGS. 16-17, according to one method of the invention, plant material, and preferably Cannabis plant material, may be harvested and frozen prior to processing. This frozen Cannabis plant material may be added to a mixing tank (5) along with a quantity of RO water and RO ice forming an organic base material. In alternative embodiments, frozen Cannabis plant material may be added to a mixing tank (5) along with a quantity of RO water, wherein the mixing tank (5) may be thermally regulated (without the use of ice) so as to maintain the temperature of the water at a desired level. In one embodiment, the mixing tank (5) may include a thermal jacket (not shown) or other refrigeration apparatus that may maintain the temperature of the water. Notably, when using RO water in the mixing tank (5), the lack of impurities removes potential nucleation sites allowing the water to be supercooled, for example through the use of applied refrigeration that can chill the RO water below the traditional freezing point of 32° F. This supercooled RO water may enhance the ability of the current system to separate the trichome structures from the plant material, thereby increasing yields and reducing run-times. The mixing tank (5) may further be insulated to prevent the transfer of thermal energy and to assist in the maintenance of a consistent temperature throughout the agitation process as generally described below.

Referring again to FIGS. 16-17, according to one method of the invention, the organic base material in the mixing tank (5) may be agitated such that sheer forces may be applied to the non-living tissue of the trichome, and in particular the narrow trichome stalk, such that the structure is separated from the plant material. In one embodiment, this agitation step may be accomplished manually, for example by one or more rotatable flywheels coupled with a series of paddles or extensions configured to agitate the organic base material in the mixing tank (5) and sheer the trichome structures from the plant material. In one embodiment, a rotatable flywheel may agitate the organic base material for between 10-30 minutes. Naturally, the movement of the flywheel may be manually operated by a user, or automatically engaged, for example through a motor-driven system.

Agitation of the organic base material may be also accomplished by introducing rotational or vibrational energy to the mixing tank (5), for example through a tank agitator (6). In this embodiment, a tank agitator (6) may include a motorized component that is in communication with the mixing tank (5) such that rotational or vibrational energy may pass from the tank agitator (6) to the mixing tank (5) with sufficient force to separate the trichome structures from the plant material. In one embodiment, a tank agitator (6) may be coupled with the mixing tank (5), while in alternative embodiments a tank agitator (6) may be indirectly coupled with the mixing tank (5). In this indirect configuration, a frame agitator (not shown) may be coupled with a support frame (23) that is in communication with the mixing tank (5).

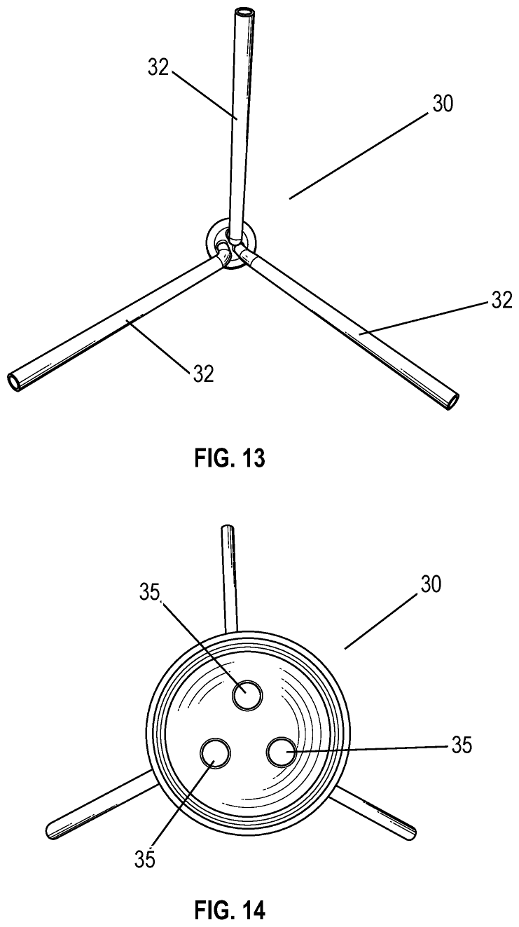

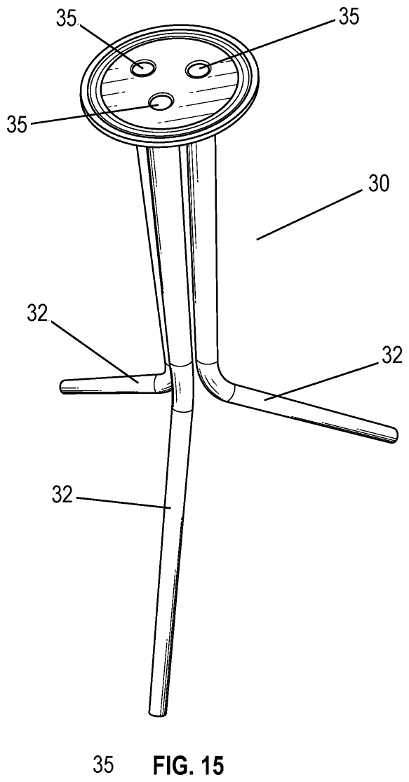

Agitation within the mixing tank (5) may be accomplished through a tank agitator (6) configured to inject or recirculate RO water through a multi-directional agitation nozzle (30). As shown in FIGS. 13-15 and 16, a multi-directional agitation nozzle (30) may be configured to be positioned inside the mixing tank (5) having a plurality of injection valves (32) and allow for the non-laminar flow of RO water into the internal compartment of the mixing tank. In this embodiment, the plurality of injection valves (32) can be positioned in opposing or equidistance angled configurations such that RO water passing through is distributed in a multi-directional fashion. This feature of the invention allows for the uniform creation of non-laminar, or turbulent water flow within the mixing tank. The creation of this uniform turbulent water flow within the mixing tank allows for sheer forces to be more efficiently and evenly applied to the plant material, causing the trichomes structures to be separated.

The level of turbulence can be regulated through the rate of RO water flow through the multi-directional agitation nozzle (30), as well as the size of the injection valve apertures (35). For example, a pump can be used to control the rate of flow through the multi-directional agitation nozzle (30). Moreover, multi-directional agitation nozzle (30) having narrower or wider injection valve apertures (35) may cause the flow rate through the nozzle to increase or decrease, respectively. The flow of RO water through the multi-directional agitation nozzle (30) may be also controlled by one or more manual or automatic valves that may decrease, increase, or stop the flow of water independently, or collectively through one or more of the injection valve apertures (35).

In one embodiment, the pump or valves in fluid communication with multi-directional agitation nozzle (30) may be manually or automatically operated in response to a signal generated by a sensor (33) transmitted to a digital device (34) having a processing system configured to effect one or more executable applications in response to the signal from one or more sensors (33). The sensor (33) of the invention may be responsive to one or more input parameters, such as the rate or quantity of RO water injected into the mixing tank (5), the level of turbulence present in the mixing tank (5) during agitation, a preset time limit, the quantity of biomass present in the mixing tank (5), temperature of the RO within the mixing tank, among other parameters. The sensor (33) of the invention may generate a signal that may be transmitted to a digital device (34) having a processing system configured to effect one or more executable applications in response to the signal from one or more sensors (33) that may affect the rate of fluid injection into the mixing tank (5). In this manner, the agitation of the plant material positioned within the mixing tank (5) may be automated and optimized based on one or more predetermined parameters.

As shown in FIG. 16, in one embodiment RO water may pass through a modular separation column (2) and be ejected as wastewater which may be responsive to a pump, such as a brewer pump, or other food/pharmaceutical grade pump that may direct the wastewater through a filter (35), such as a carbon filter, and redirect it back to the mixing tank. In this configuration of the invention, the wastewater may be continuously recirculated back to the mixing tank, through the multi-directional agitation nozzle (30). Notably, while reference is made to a modular separation column (2), in certain embodiments a modular separation column (2) may include a unitary component, or a separable multi-components column.

Again, a recirculation pump may be manually or automatically operated in response to a signal generated by a sensor (33) transmitted to a digital device (34) having a processing system configured to effect one or more executable applications in response to the signal from one or more sensors (33). The sensor (33) of the invention may be responsive to one or more input parameters, such as rate or quantity of wastewater expelled from the modular separation column (2), the rate of water flow through the modular separation column (2), the quantity of RO water present in the mixing tank, a preset time limit, temperature of the RO within the mixing tank, modular separation column (2), or wastewater among other parameters. As generally described above, the sensor (33) of the invention may generate a signal that may be transmitted to a digital device (34) having a processing system configured to effect one or more executable applications in response to the signal from one or more sensors (33) that may affect the rate of recirculation of wastewater back into the mixing tank (5). In this manner, the agitation of the plant material positioned within the mixing tank (5) by the recirculation of wastewater from the modular separation column (2) may be automated and optimized based on one or more predetermined parameters. Referring now to FIGS. 8, and 16-17, the invention may include a detritus lining (8) configured to hold the organic base material. In this embodiment, a detritus lining (8) may include a mesh insert configured to prevent the passage of large organic plant material, while allowing the passage of water and separated trichome structures present in the organic base material. In one preferred embodiment, a detritus lining (8) may include a mesh insert having a pore size of at least 220 μm or greater and may further be configured to be positioned within the mixing tank (5) during agitation of the plant material. (Naturally, this pore size is exemplary only, and should not be construed as a necessary limitation as to this preferred embodiment.) As noted above, this process may be assisted by agitation of the mixing tank (4).

Again, as generally outlined in FIGS. 16-17, in one optional embodiment, separated trichome structures, and other components of the organic base material below the pore size limit of the detritus lining (8), (which in this embodiment may be at least 220 μM), may pass through the detritus lining (8) and be optionally collected in a settling reservoir (10) positioned below the detritus lining (8). In one embodiment, a settling reservoir (10) may be a separate holding tank or structure, while in alternative embodiments it may be positioned at the bottom of the mixing tank (5). As shown in FIG. 7, a mixing tank (5) may include a feed conduit (7) that may further be controlled by a feed valve (36) that may be in fluid communication with a settling reservoir (10), or as described below, a modular separation column (2). In one alternative embodiment, the flow of organic base material through the feed conduit (7) may be facilitated by a feed pump (11). Again, this feed pump (11) may be in fluid communication with a settling reservoir (10), or as described below directly with a modular separation column (2) bypassing the settling reservoir and may facilitate the flow of the detritus-screened organic base material to one of more of these locations.

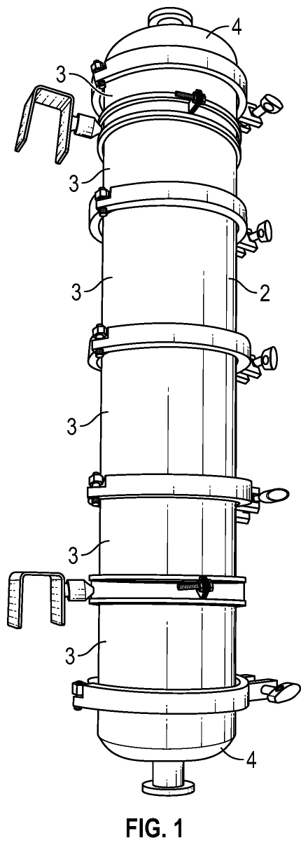

In one embodiment of the invention, the detritus-screened organic base material may be fed or pumped directly into a modular separation column (2) and undergo a series of stepwise screenings to capture and extract the separated trichome structures for later processing. Referring to FIGS. 1 and 8, a modular separation column (2) may include one or more modular casings (3) coupled at their proximal and terminal ends with an end cap (4). In one preferred embodiment, modular casings (3) and end caps (4) may be formed from stainless steel, or other sufficiently ruggedized materials such as plastic or other composites, and preferably a material that may be easily and efficiently cleaned and sterilized, such as food/pharmaceutical grade steel or other like material.

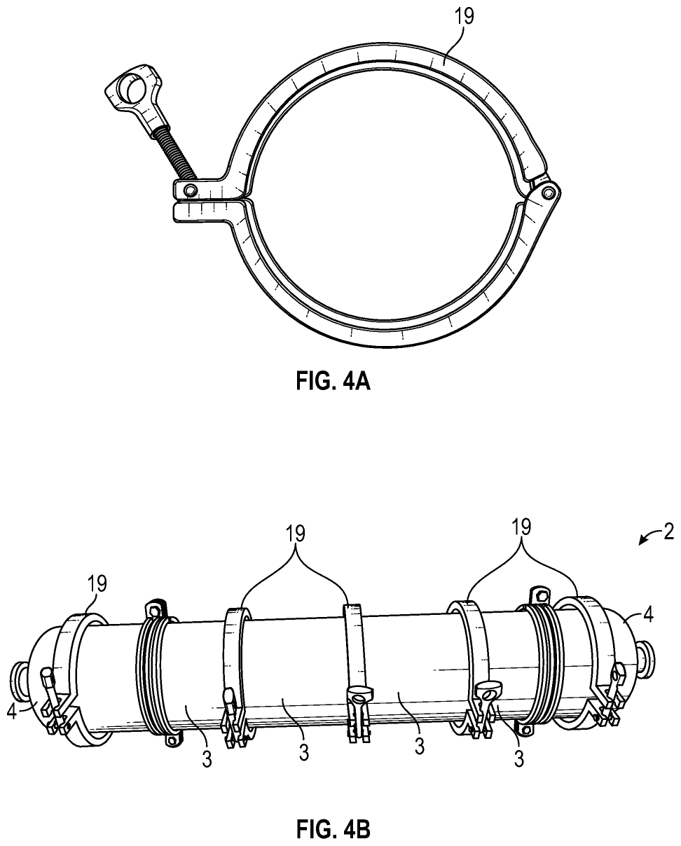

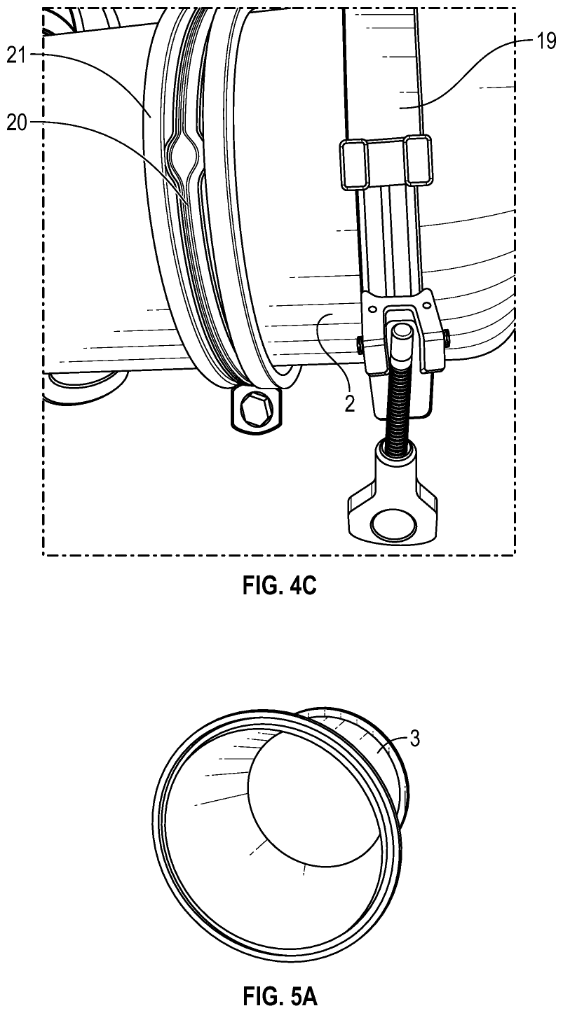

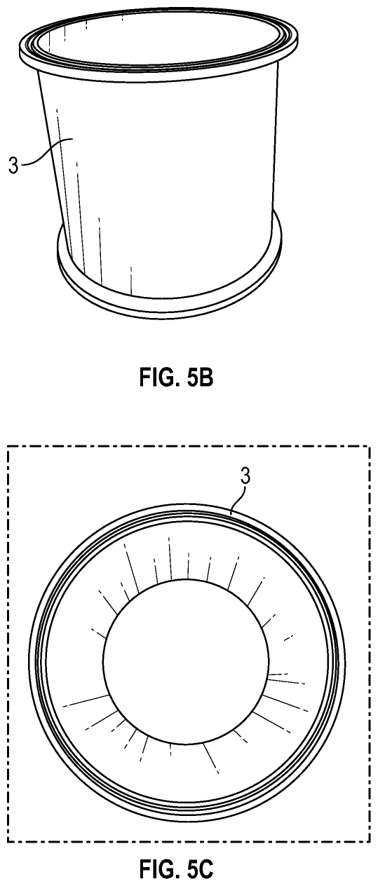

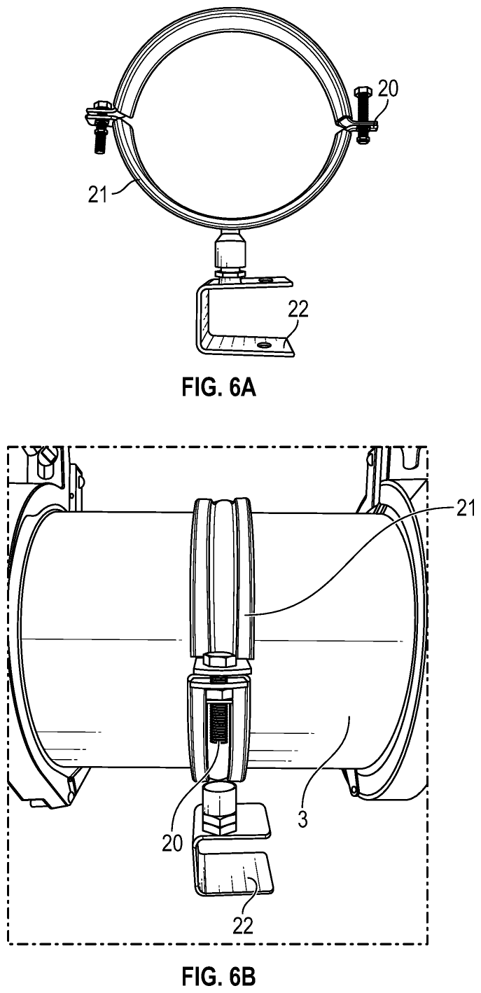

In this preferred embodiment, a modular separation column (2) may include a linear column structure formed by a plurality of modular casings (3) secured with a series of column couplers (19). As generally shown in FIGS. 4B-4C, in this embodiment, a pair of modular casings (3) may be positioned such that they form a hollow linear column structure and may further be secured in by a column coupler (19) which may be a radial fastener that is configured to be positioned over the extended rims of the modular casings (3) when placed together and form a water-tight seal.

In alternative embodiments, a modular separation column (2) may include a plurality of modular casings (3) that may be interlocked together, forming a water-tight hollow linear column structure. In this embodiment, the modular casings (3) of the invention may be configured to be coupled together without any external coupling device, such as a column coupler (19). For example, in one embodiment the modular casings (3) of the invention may be configured to have threaded interlocking coupling positions such that a plurality of modular casings (3) may be threaded with one another, forming a modular separation column (2). Still further embodiments may include integrally configured fitted couplers, such as snap couplers, slide couplers, or quick release couplers, that may further include one or more sealing components to help form a water-tight coupling between modular casings (3) or a modular casing and an end cap (4).

In another embodiment of the invention, a modular separation column (2) may include a plurality of internally positioned mesh inserts (13). As demonstrated in FIG. 8, one or more mesh inserts (13) may be positioned internally within a modular separation column (2), and preferably may be positioned such that each modular casing (3) may be associated with an individual mesh insert (13). In a preferred embodiment, a mesh insert (13) may be configured to include a mesh base (15), mesh sidewall (14) and a radial extension (16) which may be made of a mesh or non-mesh material. While any mesh material having a pore size sufficient to allow the flow of RO water through the modular separation column (2), while capturing trichome structures may be used with the invention, in a preferred embodiment, a mesh insert (13) having a metal mesh formed of food/pharmaceutical grade steel or other like material may be preferred.

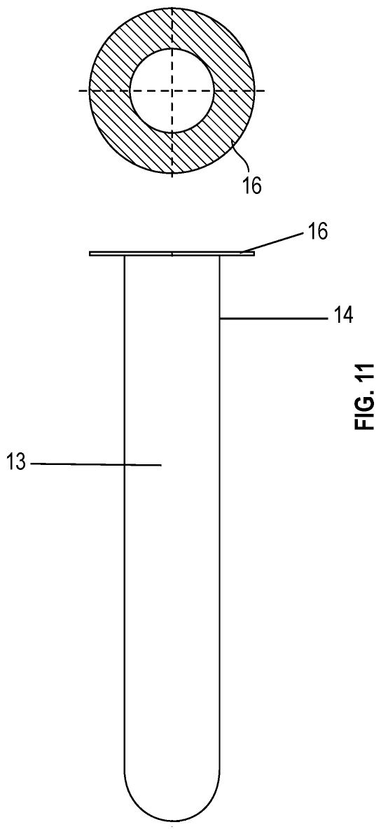

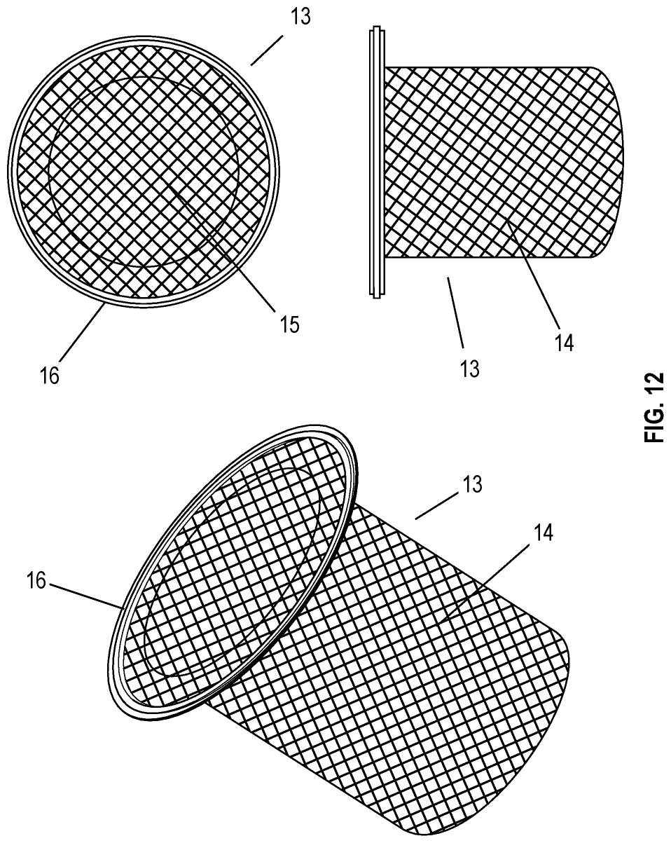

As demonstrated in FIG. 11, in this preferred embodiment the radial extension (16) may be positioned in between the mated rims of a pair of modular casings (3), or a modular casing (3) and end cap (4). In this configuration, a plurality of mesh inserts (13) may be secured along the length of the modular separation column (2), forming a closed-loop and stepwise trichome filtration and extraction system. Importantly, in this preferred embodiment, each mesh insert (13) may have a different mesh pore size. For example, the first mesh insert (13) positioned at the top, or proximal end of the column may have a larger mesh pore size that the next mesh insert (13) positioned below it, and so on.

In one embodiment, a modular separation column (2) may include one mesh insert or a plurality of sequentially secured mesh inserts (13) along the length of the column, wherein each mesh insert (13) has a smaller pore size that the prior mesh insert (13). In one embodiment, such mesh insert(s) (13) may have a pore size between 500 μM and 1 μM, while in alternative embodiments such mesh insert(s) (13) may have a pore size between 220 μM and 45 μM. Naturally, such examples are exemplary embodiments only, as the multi-staged trichome collection array (1) may incorporate one or more mesh inserts (13) and/or detritus lining(s) (8) as may be generally desired to accomplish the trichome extraction and separation purposes of the invention.

As noted in FIG. 8, in one embodiment, a modular separation column (2) may include seven sequentially secured mesh inserts (13) along the length of the column, wherein each mesh insert (13) has a smaller pore size that the prior mesh insert (13). For example, in this embodiment:
a first mesh insert (13) may have a pore size of 220 μM;
a second mesh insert (13) may have a pore size of 190 μM;
a third mesh insert (13) may have a pore size of 160 μM;
a fourth mesh insert (13) may have a pore size of 120 μM;
a fifth mesh insert (13) may have a pore size of 100 μM;
a sixth mesh insert (13) may have a pore size of 90 μM; and
a seventh mesh insert (13) may have a pore size of 45 μM.

In another preferred embodiment, a modular separation column (2) may include six sequentially secured mesh inserts (13) along the length of the column, wherein each mesh insert (13) has a smaller pore size that the prior mesh insert (13). For example, in this embodiment:
a first mesh insert (13) may have a pore size of 190 μM;
a second mesh insert (13) may have a pore size of 160 μM;
a third mesh insert (13) may have a pore size of 120 μM;
a fourth mesh insert (13) may have a pore size of 100 μM;
a fifth mesh insert (13) may have a pore size of 90 μM; and
a sixth mesh insert (13) may have a pore size of 45 μM.

In another preferred embodiment, a modular separation column (2) may include four sequentially secured metal mesh inserts (13) along the length of the column wherein each mesh insert (13) has a smaller pore size than the prior mesh insert (13). For example, in this embodiment:
a first metal mesh insert (13) may have a US standard mesh size of 60;
a second metal mesh insert (13) may have a US standard mesh size of 80;
a third metal mesh insert (13) may have a US standard mesh size of 170; and
a fourth metal mesh insert (13) may have a US standard mesh size of 325.

Notably, in this embodiment, a detritus lining (8) may be considered a mesh insert (13) and may preferably include a pore size sufficient to generally capture plant material from the base organic material as generally described herein. As can be seen from the Figures, detritus screen organic base material containing the separated trichome structures may be fed into the top of the modular separation column (2) and sequentially pass through the series of mesh inserts (13) such that a portion of separated trichome, or organic base material is captured at each mesh insert level based on its size and ability to pass through that specific mesh insert (13). Notably, as opposed to the traditional Bubble Bag™ system, because the sidewalls (14) of the mesh insert (13) allow for the flow of water through the sides of the filter, the invention's modular separation column (2) may operate as a closed-loop system that does not require a worker to continually apply water to push the material to be captured to the bottom of the filter.

Notably, this configuration also allows for the flow of organic base material to exit the sides of the mesh insert (13) and capture trichome structures—which is not possible with traditional Bubble Bag™ systems. This side-flow of organic base material allows for a more efficient flow of water through the column as it may continue to pass through the sidewall (14) of the mesh insert (13) as the bottom portion of the mesh insert (13) becomes blocked due to the accumulation of trichome structures, or other components of the organic base material. This further allows for additional processing runs to be accomplished before the mesh inserts (13) may need to be removed due to water flow blockages.

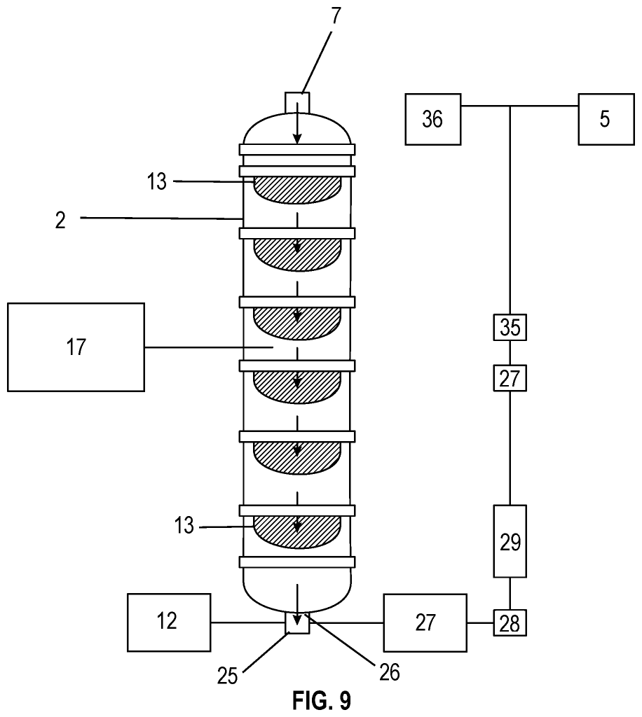

Generally referring to FIG. 9, one or more mesh inserts (13) may be positioned internally within a modular separation column (2), each insert being further positioned within at least one support mesh insert (31). In this preferred embodiment, a support mesh insert (31) may be formed of a metal mesh, having a pore size that may allow for RO water to pass through the mesh inserts (13) positioned within the modular separation column (2). In a preferred embodiment, a support mesh insert (31) may have a mesh or pore size that is larger than the corresponding mesh inserts (13) it is supporting. For example, a support mesh insert (31) may have a mesh size larger than a US standard mesh size of 60, and preferably a US standard mesh size of 25. Standard US mesh definitions are provided in Table 1 below.

In a preferred embodiment a support mesh insert (13) may be configured to include a mesh base (15), mesh sidewall (14) and a radial extension (16) which may be made of a mesh or non-mesh material. While any mesh material having a pore size sufficient to allow the flow of RO water through the modular separation column (2), while capturing trichome structures may be used with the invention, in a preferred embodiment, a mesh insert (13) having a metal mesh formed of food/pharmaceutical grade steel or other like material may be preferred.

As demonstrated in FIG. 11, in this preferred embodiment the radial extension (16) may be positioned in between the mated rims of a pair of modular casings (3), or a modular casing (3) and end cap (4). In this configuration, a plurality of mesh inserts (13) may be secured along the length of the modular separation column (2) forming a closed-loop and stepwise trichome filtration and extraction system. Importantly, in this preferred embodiment, each mesh insert (13) may have a different mesh pore size. For example, the first mesh insert (13) positioned at the top, or proximal end of the column may have a larger mesh pore size that the next mesh insert (13) positioned below it, and so on.

The modular separation column (2) of the invention may further be temperature controlled. In this embodiment, a thermal jacket (not shown), or other refrigeration device may be positioned over the modular separation column (2) to allow it to maintain a desired temperature so as to increase overall batch yields and prevent degradation of any separated trichome structures passing through the column or captured by one or more of the mesh inserts (13).

The modular separation column (2) of the invention may optionally be subject to agitation. In this embodiment, a column agitator (17) may include a motorized component that is in communication with the modular separation column (2) such that rotational or vibrational energy may pass from the column agitator (17) to the modular separation column (2) with sufficient force to assist the flow of water and capture of hash resin in the mesh inserts (13) positioned along the length of the column. This agitation may further help the mesh inserts (13) from being clogged with material impeding the flow of water through the column. In one embodiment, a column agitator (17) may be coupled with the modular separation column (2), while in alternative embodiments a column agitator (17) may be indirectly coupled with the modular separation column (2). In this indirect configuration a column agitator (17) may be coupled with a support frame (23) that is in communication with the column structure generally.

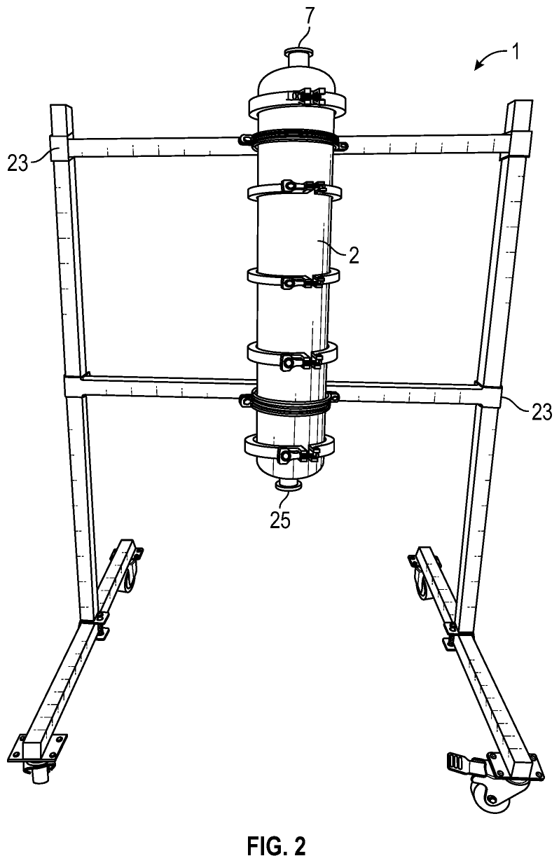

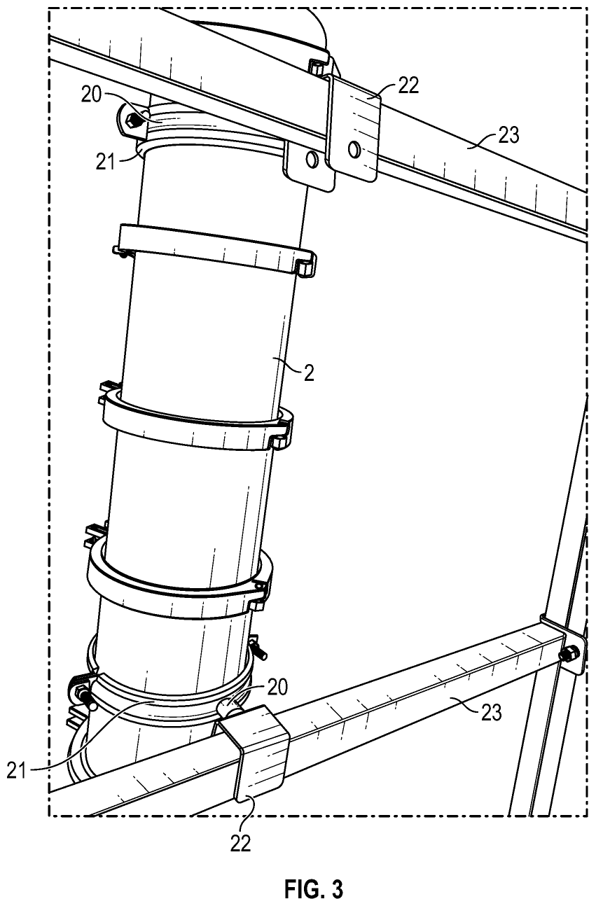

Referring now to FIGS. 2 and 3, in one embodiment a modular separation column (2) may be coupled with an adjustable support frame (23) configured to position the column in an approximately vertical position. This support frame (23) may be adjustable to accommodate different sized columns depending on the number of modular casings (3) that are used to generate the column. As further demonstrated in FIG. 3, the modular separation column (2) of the invention may be secured to an adjustable support frame (23) by one or more mounting couplers (20) having a support frame bracket (22) configured to allow the column to be suspended in a vertical orientation. In the embodiment shown in the figures, the support frame bracket (22) comprises a coupler arm configured to be secured to a horizontal bar. Additional embodiments may include a variety of coupler configurations, such as snap, slide, or even quick release coupler mechanisms that may be configured to be secured to a support frame (23) or other support surface. Notably, in this embodiment a mounting coupler (20) may include an insulated covering (21) positioned between the outer-surface of the modular casing (3) and the mounting coupler (20). This insulated covering (21) may allow for a more secure positioning of the column while reducing the risk of damaging the outer surface of the column components.

Referring now to FIGS. 1, 9, and 16, the modular separation column (2) of the invention may include one or more release pipes to allow the organic base material, generally referred to as wastewater not captured by the mesh insert(s) (13) to exit the column and be captured by a wastewater collection container (36) or expelled into an appropriate material handling system. In this preferred embodiment, the terminal end cap of the column may include a release pipe (25) that may further include a release valve (25) to control the flow of material from the column. In another preferred embodiment, a release pipe (25) may be coupled with a recovery pump that may actively draw fluid, in this case the uncaptured organic base material, from the column to be discarded or recirculated through a recirculation valve (28) and/or recirculation pipe (29) configured to route the uncaptured organic base material from the column back to the mixing tank (5), through a multi-directional agitation nozzle (30), or the top of the modular separation column (2). In still further embodiments, a vacuum pump may be used to generate a vacuum environment inside the modular separation column (2) such that organic base material may be pulled through the column more efficiently. As noted above, the circulation, or recirculation of fluid through the inventive system may be manually or automatically operated in response to a signal generated by a sensor (33) transmitted to a digital device (34) having a processing system configured to effect one or more executable applications in response to the signal from one or more sensors (33).

As noted above, plant material may undergo one or more processing cycles to remove trichome structures. For example, in a preferred embodiment, a first quantity of plant material may be processed by the multi-staged trichome collection array (1) described above. After this first cycle is complete, the plant material may undergo a second, or even third processing cycle. In a preferred embodiment, prior to initiating any subsequent cycle, the trichome structures captured by the mesh inserts (13) in the inside of the modular separation column (2) may be removed and further processed into hash resin for commercial or therapeutic applications. In between each processing run, the system, including the mixing tank (5) and modular separation column (2) may be cleaned and/or sterilized in preparation for a new processing run.

Notably, individual mesh insert (13) may capture a differentially sized trichome structures, with the largest being caught by the upper mesh inserts (13) having the largest pore size, while smaller, more immature trichome structures may be captured in lower mesh inserts (13) having a smaller pore size. In this configuration, each mesh insert may contain a unique ratio of trichome phytochemical constituents. (See e.g., Livingston al. (2020), Cannabis glandular trichomes alter morphology and metabolite content during flower maturation. Plant J, 101: 37-56.)

For example, bulbous trichomes, generally being the smallest, may be captured in a terminal mesh insert (13) having a small pore size. Capitate-sessile trichomes, being generally larger than bulbous trichomes may be caught by one or more discrete middle positioned mesh inserts (13), while capitate-stalked trichomes, being the most abundant and largest type of trichome found in Cannabis may be captured in a proximal mesh insert (13) at the top of the modular separation column (2). Again, as noted above, each discrete mesh resin may include a trichome population having a unique phytochemical profile such that the ratios of cannabinoids, endocannabinoids, terpenes and even flavonoids may have individually desirable commercial or therapeutic characteristics.

In certain embodiments, the inventive technology may employ a single multi-staged trichome collection array (1) to separate and extract trichome structures, while in additional embodiments, a plurality of multi-staged trichome collection arrays (1) may be positioned in series, or in parallel, and used to separate and extract trichome structures. For example, in one embodiment, a plurality of modular separation columns (2) may be in fluid communication with a mixing tank and may simultaneously, or sequentially process base organic material fed into these respective columns. In alternative embodiments, a plurality of modular separation columns (2) may be in fluid communication with one another and a mixing tank, such that the system may sequentially process base organic material passed through a series of columns.

It will be understood by all readers of this written description that the example embodiments described herein and claimed hereafter may be suitably practiced in the absence of any recited feature, element or step that is, or is not, specifically disclosed herein. For instance, references in this written description to “one embodiment,” “an embodiment,” “an example embodiment,” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described. No language or terminology in this specification should be construed as indicating any non-claimed element as essential or critical. All methods described herein can be performed in any suitable order unless otherwise indicated herein. The use of any and all examples, or example language (e.g., “such as”) provided herein, is intended merely to better illuminate example embodiments and does not pose a limitation on the scope of the claims appended hereto unless otherwise claimed.

Throughout this specification (i.e., the written description, drawings, claims and abstract), the word “comprise”, or variations such as “comprises” or “comprising, “including,” “containing,” and the like will be understood to imply the inclusion of a stated element or integer or group of elements or integers but not the exclusion of any other element or integer or group of elements or integers, unless the context requires otherwise.

To facilitate understanding of this example embodiments set forth herein, a number of terms are defined below. Generally, the nomenclature used herein and the laboratory procedures in biology, biochemistry, organic chemistry, medicinal chemistry, pharmacology, etc. described herein are generally well known and commonly employed in the art. Unless defined otherwise, all technical and scientific terms used herein generally have the same meaning as commonly understood in the art to which this disclosure belongs. In the event that there is a plurality of definitions for a term used herein, those in this written description shall prevail unless stated otherwise herein.

As used herein, “Cannabis” refers to a genus of flowering plants that includes a single species, Cannabis sativa, which is sometimes divided into two additional species, Cannabis indica and Cannabis ruderalis. These three taxa are indigenous to Central Asia, and South Asia. Cannabis has long been used for fiber (hemp), for seed and seed oils, for medicinal purposes, and as a recreational drug. Various extracts including hashish and hash oil are also produced from the plant. Suitable strains of Cannabis include, e.g., indica-dominant (e.g., Blueberry, BC Bud, Holland's Hope, Kush, Northern Lights, Purple, and White Widow), Pure sativa (e.g., Acapulco Gold and Malawi Gold (Chamba)), and Sativa-dominant (e.g., Charlotte's Web, Diesel, Haze, Jack Herer, Shaman, Skunk, Sour, and Te Puke Thunder). The Cannabis plant can include any physical part of the plant material, including, e.g., the leaf, bud, flower, trichome, seed, or combination thereof. Likewise, the Cannabis plant can include any substance physically derived from Cannabis plant material, such as, e.g., kief and hashish.

As used herein, “trichome” refers to a fine outgrowth or appendage on plants and certain protists. They are of diverse structure and function. In reference to Cannabis, the trichome is a glandular trichome that occurs most abundantly on the floral calyxes and bracts of female plants.

As used herein, “hash” or “hash resin” refers to a Cannabis product composed of preparations of stalked resin glands, generally referred to as trichomes, which may further be compressed or purified. It contains the same active ingredients—such as THC and other cannabinoids—but in higher concentrations than, for example, unsifted buds or leaves.

As used herein, a “cannabinoid” is a chemical compound (such as cannabinol, THC or cannabidiol) that is found in the plant species Cannabis among others like Echinacea; Acmella Oleracea; Helichrysum Umbraculigerum; Radula Marginata (Liverwort) and Theobroma Cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference); (3 S,4R)-7-hydroxy-Δ6-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]hept-2-en, and other 4-phenylpinene derivatives disclosed in U.S. Pat. No. 5,434,295, which is incorporated by reference; and cannabidiol (−)(CBD) analogs such as (−)CBD-monomethylether, (−)CBD dimethyl ether; (−)CBD diacetate; (−)3′-acetyl-CBD monoacetate; and ±AF11, all of which are disclosed in Consroe et al., J. Clin. Phannacol. 21:428S-436S, 1981, which is also incorporated by reference. Many other cannabinoids are similarly disclosed in Agurell et al., Pharmacol. Rev. 38:31-43, 1986, which is also incorporated by reference.

Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol,trihydroxy-delta-9-tetrahydrocannabinol, and cannabinol. Examples of cannabinoids within the context of this disclosure include tetrahydrocannabinol and cannabidiol.

The term “endocannabinoid” refer to compounds including arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),1 1 (Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA). Particularly preferred endocannabinoids are AEA, 2-AG, 1-AG, and DHEA.

Terpenoids a.k.a. isoprenoids, are a large and diverse class of naturally occurring organic chemicals similar to terpenes, derived from five-carbon isoprene units assembled and modified in a number of varying configurations. Most are multi-cyclic structures that differ from one another not only in functional groups but also in their basic carbon skeletons. Terpenoids are essential for plant metabolism, influencing general development, herbivory defense, pollination and stress response. These compounds have been extensively used as flavoring and scenting agents in cosmetics, detergents, food and pharmaceutical products. They also display multiple biological activities in humans, such as anti-inflammatory, anti-microbial, antifungal and antiviral. When terpenes are modified chemically, such as by oxidation or rearrangement of the carbon skeleton, the resulting compounds are generally referred to as “terpenoids.” The structure of terpenes are built with isoprenes, which are 5 carbon structures. Flavonoids are generally considered to be 15 carbon structures with two phenyl rings and a heterocyclic ring. So, there could be an overlap in which a flavonoid could be considered a terpene. However, not all terpenes could be considered flavonoids. As used herein, the terms “terpene” and “terpenoid” are used interchangeably.

Within the context of the inventive technology, the term terpene includes: Flemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. Derivatives of terpenes include Terpenoids in their forms of hemiterpenoids, monoterpenoids, sesquiterpenoids, sesterterpenoid, sesquarterpenoids, tetraterpenoids, Triterpenoids, tetraterpenoids, Polyterpenoids, isoprenoids, and steroids. They may be forms: α-, β-, γ-, oxo-, isomers, or combinations thereof.

Cannabis terpenoid profiles define the aroma of each plant and share the same precursor (geranyl pyrophosphate) and the same synthesis location (glandular trichomes) as phytocannabinoids. The terpenoids most commonly found in Cannabis extracts include: limonine, myrcene, alpha-pinene, linalool, beta-caryophyllene, caryophyllene oxide, nerolidol, and phytol. Terpenoids are mainly synthesized in two metabolic pathways: mevalonic acid pathway (a.k.a. HMG-CoA reductase pathway, which takes place in the cytosol) and MEP/DOXP pathway (a.k.a. The 2-C-methyl-D-erythritol 4-phosphate/1-deoxy-D-xylulose 5-phosphate pathway, non-mevalonate pathway, or mevalonic acid-independent pathway, which takes place in plastids). Geranyl pyrophosphate (GPP), which is used by Cannabis plants to produce cannabinoids, is formed by condensation of dimethylallyl pyrophosphate (DMAPP) and isopentenyl pyrophosphate (IPP) via the catalysis of GPP synthase. Alternatively, DMAPP and IPP are ligated by FPP synthase to produce farnesyl pyrophosphate (FPP), which can be used to produce sesquiterpenoids. Geranyl pyrophospliate (GPP) can also be converted into monoterpenoids by limonene synthase.

Some examples of terpenes, and their classification, are as follows. Hemiterpenes: Examples of hemiterpenes, which do not necessarily have an odor, are 2-methyl-1,3-butadiene, hemialboside, and hymenoside. Monoterpenes: pinene, a-pinene, β-pinene, cis-pinane, trans-pinane, cis-pinanol, trans-pinanol (Erman and Kane (2008) Chem. Biodivers. 5:910-919), limonene; linalool; myrcene; eucalyptol; a-phellandrene; β-phellandrene; a-ocimene; β-ocimene, cis-ocimene, ocimene, Δ-3-carene; fenchol; sabinene, borneol, isoborneol, camphene, camphor, phellandrene, a-phellandrene, a-terpinene, geraniol, linalool, nerol, menthol, myrcene, terpinolene, a-terpinolene, β-terpinolene, γ-terpinolene, Δ-terpinolene, α-terpineol, and trans-2-pinanol. Sesquiterpenes: caryophyllene, caryophyllene oxide, humulene, a-humulene, a-bisabolene; β-bisabolene; santalol; selinene; nerolidol, bisabolol; a-cedrene, β-cedrene, β-eudesmol, eudesm-7(1 1)-en-4-ol, selina-3,7(1 1)-diene, guaiol, valencene, a-guaiene, β-guaiene, Δ-guaiene, guaiene, farnesene, a-farnesene, β-farnesene, elemene, a-elemene, β-elemene, γ-elemene, Δ-elemene, germacrene, germacrene A, germacrene B, germacrene C, germacrene D, and germacrene E. Diterpenes: oridonin, phytol, and isophytol. Triterpenes: ursolic acid, oleanolic acid. Terpenoids, also known as isoprenoids, are a large and diverse class of naturally occurring organic chemicals similar to terpenes, derived from five-carbon isoprene units assembled and modified in a number of ways. Most are multicyclic structures that differ from one another not only in functional groups but also in their basic carbon skeletons. Plant terpenoids are used extensively for their aromatic qualities.

The term “plant” or “plant system” includes whole plants, plant organs, progeny of whole plants or plant organs, embryos, somatic embryos, embryo-like structures, protocorms, protocorm-like bodies (PLBs), and culture and/or suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The invention may also include Cannabaceae and other Cannabis strains, such as hemp, and C. indica, C. sativa generally.

As used herein, the singular forms “a,” “an,” and “the” may also refer to plural articles, i.e., “one or more,” “at least one,” “and/or” are open-ended expressions that are both conjunctive and disjunctive in operation. For example, the term “a cannabinoid” includes “one or more cannabinoids”. Further, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “a” or “an” entity refers to one or more of that entity. As such, the terms “a” (or “an”), “one or more” and “at least one” can be used interchangeably herein.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller subranges are also included. The upper and lower limits of these smaller ranges are also included therein, subject to any specifically excluded limit in the stated range. For example, a range of “about 0.1% to about 5%” or “about 0.1% to 5%” may be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “about” or “approximately” means an acceptable error for a particular recited value, which depends in part on how the value is measured or determined. In certain embodiments, “about” can mean 1 or more standard deviations. When the antecedent term “about” is applied to a recited range or value it denotes an approximation within the deviation in the range or value known or expected in the art from the measurement's method. For removal of doubt, it shall be understood that any range stated in this written description that does not specifically recite the term “about” before the range or before any value within the stated range inherently includes such term to encompass the approximation within the deviation noted above.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

TABLE 1
Particle Size Conversion Table
Sieve Designation

| Standard | Mesh | |
|---|---|---|
| 25.4 mm | 1 | in. |
| 22.6 mm | ⅞ | in. |
| 19.0 mm | ¾ | in. |
| 16.0 mm | ⅝ | in. |
| 13.5 mm | 0.530 | in. |
| 12.7 mm | ½ | in. |
| 11.2 mm | 7/16 | in. |
| 9.51 mm | ⅜ | in. |
| 8.00 mm | 5/16 | in. |
| 6.73 mm | 0.265 | in. |
| 6.35 mm | ¼ | in. |

| | |
|---|---|
| 5.66 mm | No. 3 ½ |
| 4.76 mm | No. 4 |
| 4.00 mm | No. 5 |
| 3.36 mm | No. 6 |
| 2.83 mm | No. 7 |
| 2.38 mm | No. 8 |
| 2.00 mm | No. 10 |
| 1.68 mm | No. 12 |
| 1.41 mm | No. 14 |
| 1.19 mm | No. 16 |
| 1.00 mm | No. 18 |
| 0.841 mm | No. 20 |
| 0.707 mm | No. 25 |
| 0.595 mm | No. 30 |
| 0.500 mm | No. 35 |
| 0.420 mm | No. 40 |
| 0.354 mm | No. 45 |
| 0.297 mm | No. 50 |
| 0.250 mm | No. 60 |
| 0.210 mm | No. 70 |
| 0.177 mm | No. 80 |
| 0.149 mm | No. 100 |
| 0.125 mm | No. 120 |
| 0.105 mm | No. 140 |
| 0.088 mm | No. 170 |
| 0.074 mm | No. 200 |
| 0.063 mm | No. 230 |
| 0.053 mm | No. 270 |
| 0.044 mm | No. 325 |
| 0.037 mm | No. 400 |

1-28. (canceled)

29. A system for extracting trichome structures from plant material comprising:
a mixing tank having a quantity of Cannabis plant material and a quantity of thermally controlled water;
a tank agitator adapted to separate trichome structures from said Cannabis plant material;
a multi-staged trichome collection array in fluid communication with said mixing tank, and further comprising a modular separation column having a plurality of mesh inserts positioned in series along the length of said modular separation column wherein each mesh insert has a smaller pore size that the prior mesh insert; and
a recirculation pump configured to recirculate wastewater exiting the column back to said mixing tank.

30-55. (canceled)

56. An apparatus for extracting trichome structures from plant material comprising:
a multi-staged trichome collection array comprising a modular separation column having a plurality of mesh inserts positioned in series along the length of said modular separation column wherein each mesh insert has a smaller pore size than the prior mesh insert, and wherein said modular separation column is configured to allow a mixture of water and trichome structures from Cannabis plant material to pass through the length of the column and be collected by said plurality of mesh inserts.

57. The apparatus of claim 56, further comprising a mixing tank having a quantity of Cannabis plant material and a quantity of thermally controlled water.

58. The apparatus of claim 57, further comprising a tank agitator positioned within said mixing tank and adapted to separate trichome structures from said Cannabis plant material.

59. The apparatus of claim 58, further comprising a recirculation pump configured to recirculate wastewater exiting the column back to said mixing tank.

60. The apparatus of claim 57, wherein said mixing tank is in fluid communication with said modular separation column forming a closed-loop apparatus.

61. The apparatus of claim 60, wherein said mixing tank is in fluid communication with said modular separation column through a fluid conduit and responsive to a feed pump.

62-63. (canceled)

64. The apparatus of claim 57, and further comprising a detritus lining positioned within said mixing tank wherein the lining includes a pore size that prevents Cannabis plant material from being introduced to said modular separation column, while allowing separate trichome structures in said quantity of thermally controlled water to be introduced to said modular separation column.

65. (canceled)

66. The apparatus of claim 56, wherein said water comprises a quantity of thermally controlled water having undergone reverse-osmosis forming RO water.

67. (canceled)

68. The apparatus of claim 58, wherein said tank agitator comprises a multi-directional agitation nozzle configured to inject water into said mixing tank generating a non-laminar flow of liquid within said mixing tank.

69. (canceled)

70. The apparatus of claim 56, wherein the plurality of mesh inserts comprises one or more metal mesh inserts having a pore size between 325 US standard mesh and 60 US standard mesh.

71. The apparatus of claim 56, wherein the mesh insert comprises a sidewall and base formed from a metal mesh material having a pore size between 325 US standard mesh and 60 US standard mesh.

72-73. (canceled)

74. The apparatus of claim 56, and further comprising a plurality of metal mesh inserts positioned within said modular separation column, each securing at least one of said mesh inserts.

75. The apparatus of claim 74, wherein said mesh insert has a pore size of 25 US standard mesh.

76. The apparatus of claim 56, and further comprising a filter adapted to filter the wastewater exiting the column prior to being injected into said mixing tank.

77. The apparatus of claim 57, wherein said mixing tank and said multi-staged trichome collection array form a closed-loop apparatus.

78. The apparatus of claim 77, wherein said closed-loop apparatus comprises a closed-loop apparatus having a vacuum force directing the flow of the water through the apparatus.

79. The apparatus of claim 56, wherein said modular separation column comprises a series of modular casings coupled together by one of more column couplers and a proximal and terminal end cap coupled to the proximal and terminal modular casing by one of more column couplers.

80. The apparatus of claim 79, wherein said mesh inserts or said metal mesh inserts are secured at the juncture between two modular casings, or a modular casing and an end-cap.

81-85. (canceled)

86. An apparatus for extracting trichome structures from plant material comprising:
a modular separation column securing a mesh insert positioned having a pore size that is configured to prevent trichome structures from passing through the insert, and wherein said modular separation column is configured to allow a mixture of water and trichome structures from Cannabis plant material to pass through the length of the column and be collected by said mesh inserts.

87. (canceled)
